(19) United States
(12) Patent Application Publication
(10) Pub. No.: US 2018/0093095 A1
(43) Pub. Date: Apr. 5, 2018

(54) USER INTERFACE FOR USE WITH AN ELECTROSTIMULATION SYSTEM

(71) Applicant: WEARABLE LIFE SCIENCE GMBH, Nürnberg (DE)

(72) Inventors: Philipp G. SCHWARZ, Frankfurt (DE); Nordin KOUACHE, Frankfurt (DE); Paul SYRNICKI, Regensburg (DE)

(21) Appl. No.: 15/552,003

(22) PCT Filed: Feb. 18, 2016
(86) PCT No.: PCT/EP2016/053473

§ 371 Date: Aug. 18, 2017

(30) Foreign Application Priority Data
Feb. 18, 2015 (DE) 20 2015 001 313.9
Feb. 27, 2015 (DE) 10 2015 002 484.1
Feb. 27, 2015 (DE) 10 2015 002 565.1
Aug. 14, 2015 (DE) 20 2015 005 645.8
Feb. 12, 2016 (EP) PCT/EP2016/000237

(51) Int. Cl. A61N 1/36; A61N 1/372; A61N 1/04

(52) U.S. Cl. CPC A61N 1/36003; A61N 1/0484; A61N 1/0452; A61N 1/37247

(57) ABSTRACT

A user interface for use together with an electrostimulation system, the user interface having a communication module and fasteners for fastening the communication module to the back of the hand of a person training with the system. The communication module has at one display for displaying an actual or a nominal condition of electrostimulation and/or an input device for starting or terminating electrostimulation and/or for setting parameters of the electrostimulation.

### FIELD OF THE INVENTION

The invention relates to a user interface for use with an electrostimulation system.

### BACKGROUND OF THE INVENTION

Electro-muscle stimulation (EMS) is widely known. Electrical signals and stimuli are used to stimulate a section of muscle or nerves. This serves both for muscular strengthening and for restoring movement functions.

What is important here is that the user is given the opportunity to obtain feedback about the state of the system, as well as the opportunity to vary parameters. In known stationary systems, the user is connected to the device, which is installed in an immobile fashion, via cables and can obtain and give said feedback at the device.

### SUMMARY OF THE INVENTION

It is an object of the present invention to provide an ergonomic user interface for an EMS system which is suitable in particular for mobile use. The system is intended to be usable simply and ergonomically for the user.

This object is achieved by means of the features of claim 1. The dependent claims relate to preferred embodiments.

A user interface for use with an electro-muscle stimulation system comprises a communication module and securing means for securing the communication module on the back of the hand of a person training with the system. In this case, the communication module comprises at least one display for displaying an actual or a setpoint condition of the electrostimulation and/or input means for starting or ending an electrostimulation and/or setting parameters of the electrostimulation. The positioning of the communication module on the back of the hand has some advantages. This area is readily visible to the user. In the case of a use of the EMS in the form of a mobile system, the user can use the latter outdoors and wear a jacket in the process. Consequently a communication module worn as a wristwatch, for example, is less helpful since it can easily be concealed by the sleeve and the user has to roll the sleeve back in a separate movement until he/she obtains access to the communication module. Moreover, the communication module can comprise haptic feedback means for generating mechanical, thermal or electrical stimuli. The electrical stimuli differ from EMS pulses since they do not have the objective of bringing about a muscle stimulation, but rather a pulse perceptible by nerves. On account of the large number of nerves in the hand, a user can perceive such a haptic signal significantly better than a signal that would be caused by a wristwatch-like unit. In this regard, e.g. a vibration emitted by the user interface directly covers the whole hand and is also perceived by the sensory cells provided for the fingers.

A thumb loop is suitable as a means of securing the user interface. Since the movement of the thumb is not very important in many types of sport, this loop can be used without thereby giving the user a disturbing sensation.

Alternatively or additionally a finger loop can be provided. A finger within the meaning of this document is one of the following fingers, namely index finger, middle finger, ring finger, little finger, wherein the thumb is understood not to be a finger. Each finger loop is provided for exactly one finger. It is also possible to use a finger loop for a plurality of fingers, such as e.g. two adjacent fingers, such as e.g. middle finger and ring finger or index finger and middle finger. The combination can enable a good securing of the user interface on the back of the hand.

Furthermore, alternatively or additionally, at least one holding clip can be provided, which is produced from a flexurally stiff material and is secured at least indirectly on the communication module and in this case is configured to encompass part of the flat of the hand in order to secure the user interface. Precisely when the EMS system is used in a mobile application, it is pleasant for the user to have the hands free. Consequently, any component that covers the insides of the fingers or the palm of the hand may be perceived as unpleasant. If the holding clips press as a hook from the back of the hand onto the palm of the hand, then this unpleasant sensation is minimized. The holding clip can be e.g. a wire or a stiff plastic arch and can be embodied in a flat fashion. The thickness can be less than 1 mm.

Alternatively and additionally, the securing means can comprise a wristband, which is guidable around the wrist in particular with an elastic press-on force and is preferably configured not to cover the ball of the thumb or to cover it only to the extent of 10%. The wrist has a smaller circumference than the flat of the hand. Consequently, a band can be secured here in a simple manner. If, moreover, means are provided which guide and hold the user interface in the direction of the front part of the hand, this results in a secure positioning of the user interface on the back of the hand.

Moreover, the securing means can comprise a band for the ball of the thumb, which is guidable around the hand in particular with an elastic press-on force. By virtue of the elastic press-on force, it is furthermore possible to use the hand without restriction. Unlike a wristband, a band for the ball of the thumb practically does not limit the mobility of the wrist. Precisely in conjunction with a thumb loop, the user interface is secured non-displaceably on the back of the hand.

On the one hand, the wristband and/or band for the ball of the thumb can be designed such that they/it can open and close. On the other hand, they/it can consist of a highly elastic material (elastic extensibility >60%) and be embodied as a closed loop.

It is preferred in many cases for at least 60%, in particular at least 80%, of the palm of the hand not to be covered by the securing means. The palm of the hand does not comprise the inner surface region defined by the fingers or by the thumb. In this regard, the haptics of the hand are restricted as little as possible.

Moreover, additionally or alternatively, securing means can be part of a garment for covering the forearm of a person using the user interface. Also the communication module can be connectable via the securing means, in particular a hook-and-loop connection, to a garment for covering the forearm of a person using the user interface. In this case, said garment is preferably a shirt having a thumb loop and/or finger loop. By way of example, said garment as securing means can have a pocket in which the communication means is accommodated.

Furthermore, it is preferred if the communication module comprises at least two electronic components, such as, in particular a display, input switch, output luminaire(s), such as, in particular, LED, and/or an energy supply, and a relative movement, in particular a pivoting movement, of these components with respect to one another is possible.

In particular, said at least two components are connected to the user interface in a non-removable manner. In particular, all of said two components are securable on the back of the hand of the person training with the system.

Furthermore, embodiments are preferred, in particular, in which the user interface does not comprise a glove, does not comprise a mitten and does not comprise a fingerless glove. By this means too, the haptics of the hand are only minimally restricted.

Furthermore, it is possible for a band having a thickness of max. 2 cm, preferably max. 1 cm, to run over the flat of the hand and the back of the hand and for the thumb loop to fix the band.

In one embodiment of the system according to the invention, the user interface, in particular the visualization unit and/or the input means, can be secured on a band that can be worn on the hand. In this case, it is preferred for the band to be configured such that it runs over the flat of the hand and back of the hand and is equipped with a thumb loop. The thumb loop fixes the band. As a result, it is possible for the user interface to be positioned in a readily visible manner even if a textile comprised by the system, in particular a garment—as is often the case with sports clothing—has comparatively long sleeves. The band can be provided with a hook-and-loop fastener for individual adaptation.

Moreover, the stimulation unit can be connected in a wireless and/or wired manner to the control unit, which preferably clarifies the stimulation on the hand or the wrist in particular signals the change between pulse and pause; this can give signals by means of an acoustic and/or visual and/or haptic stimulus (vibrating), and at the same time serve for controlling the intensity and/or display signals via LEDs. Said stimulation unit can be secured to the thumb via an armband that is not exactly an armband, but rather can be worn around the back of the hand, preferably by means of a loop.

Gesture control is possible, wherein the user interface comprises an inclination sensor, a compass and/or an acceleration sensor and is configured to measure angular positions and/or movements and/or accelerations. Inputs or commands of the user can be identified from these measurement values by the control in the sense of gesture control.

### BRIEF DESCRIPTION OF THE DRAWINGS

Exemplary embodiments of the present invention are explained in greater detail below with reference to the accompanying drawings and examples. In the figures:

FIG. 1 shows a schematic illustration of a portable system for controlling EMS pulses during an EMS application on an EMS user,

FIG. 2 shows a schematic illustration of a system for controlling stimulation pulses with at least two electrodes and a conductor for the electrical connection of pulse unit and electrode,

FIG. 3 shows a schematic illustration of an EMS user when performing a movement sequence which is detected by means of a sensor and visualized on a monitor, as a virtual reality application,

FIG. 4 shows a schematic illustration of an EMS user who is equipped with at least two electrodes,

FIG. 5 shows an illustration of a voltage profile of a stimulation pulse,

FIGS. 6, 7 show one embodiment of a user interface with holding clips for securing on a hand,

FIG. 8 shows a variant of the user interface comprising a communication module divided in two,

FIGS. 9, 10 show one embodiment of the user interface comprising a finger loop and a wristband,

FIGS. 11, 12 each show views of an embodiment with a band for the ball of the thumb, and

FIG. 13 shows the user interface from FIGS. 11 and 12 in an opened view.

### DETAILED DESCRIPTION OF THE INVENTION

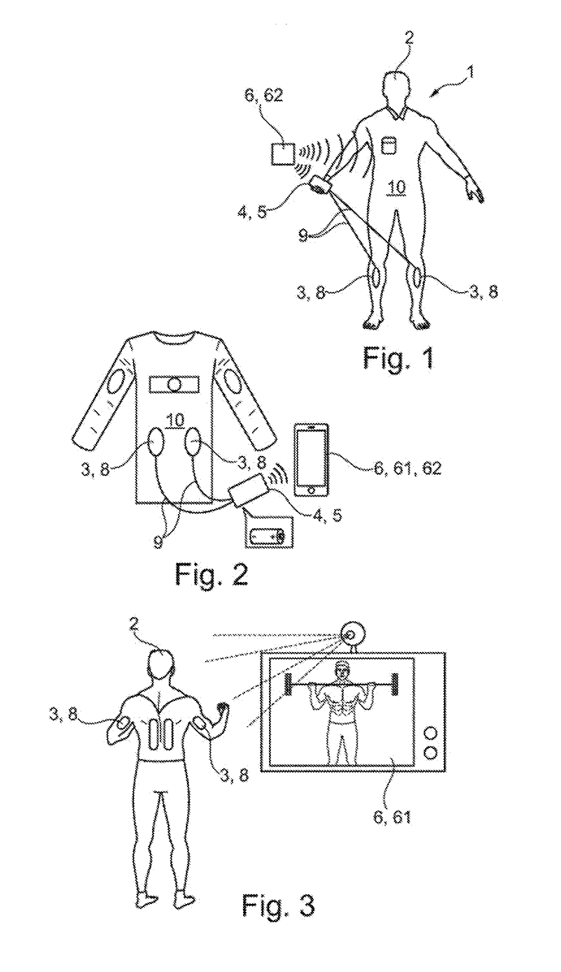

FIG. 1 shows a schematic illustration of control of stimulation pulses. The system 1 for controlling stimulation pulses during a stimulation on a user 2 comprises at least one sensor 3, a data processing unit 4 and a pulse unit 5. In the embodiment illustrated in FIG. 1, the electrodes 8 and the sensors 3 are connected to a textile, here a training suit 10, and are fixedly attached in each case in a lower leg region of the training suit 10. As a result, a portable system 1 is provided which allows the user to carry out the stimulation application without restriction in said user's freedom of movement and/or spatially. In this case, the sensor 3 is e.g. suitable for measuring a measurement value, in particular the EMG activity of the user 2. This advantageously allows an EMG activity of the user 2 to be measured and a stimulation pulse, in particular an EMS pulse, to be triggered which is varied in one or more stimulation pulse parameters depending on the measurement value or control signal. Advantageously, one or more sensors 3 of the same type or different types can be arranged in the system 1.

The data processing unit 4 is configured to compare the measurement value with a threshold value and to generate a control signal to the pulse unit 5 if the measurement value and the threshold value are in a predefineable ratio to one another. In the embodiment shown in the present case, pulse unit 5 and data processing unit 4 are fitted in a common housing which can be carried in one hand by the user 2, or optionally can be inserted into a pocket or connected to the training suit 10 in a releasable manner. In this case, the pulse unit 5 is suitable for triggering stimulation pulses and is configured to vary one or more stimulation pulse parameters depending on the control signal.

A method in which a pulse unit triggers one or more stimulation pulses comprises at least the following steps: a) measuring a measurement value, b) comparing the measurement value with a threshold value, c) generating a control signal if the measurement value and the threshold value are in a predefineable ratio to one another, and d) varying a stimulation pulse parameter depending on the control signal.

In this case, the measurement value measured by means of a sensor is compared with a threshold value by means of suitable algorithms. Such an algorithm can advantageously be predefined or adjustable or predefineable in the data processing unit. If it is ascertained that the measurement value and the threshold value are in a predefined ratio to one another, a corresponding control signal is generated and a pulse parameter is varied depending on the control signal. A corresponding stimulation pulse having a changed pulse parameter can then be triggered by the pulse unit. Consequently, e.g. the stimulation pulse intensity can be increased or decreased depending on the measurement value. Likewise, alternatively or additionally, further stimulation pulse parameters such as pulse type, intensity, duration of the stimulation pulse, frequency, ramp, pulse pause, individual pulse width and/or individual pulse duration can be varied.

The system 1 illustrated in FIG. 1 additionally comprises a user interface 6, comprising an input means 62, e.g. keys. In the embodiment illustrated, the user interface 6 is arranged in a housing separate from the data processing unit 4 and pulse unit 5, and is configured as a remote control. Thus, by means of the remote control comprising the user interface 6, the data processing unit 4 and the pulse unit 5 can be controlled and adjusted, without the user 2 having to carry the remote control with him/her during the stimulation application. The portable housing comprising the data processing unit 4 and the pulse unit 5 furthermore comprises an energy source 7.

As evident from FIG. 2, the textile 10 can also be configured as a top. Here the electrodes 8 and sensors 3 are arranged in each case in a left and right region of the stomach. Furthermore, the embodiment illustrated in FIG. 2 differs from the embodiment illustrated in FIG. 1 in that a cellular phone or tablet PC is used as visualization unit 61 and input means 62. In this case, the transmission of data from the visualization unit 61 and input means 62 to the data processing unit 4 is effected by means of suitable transmission means, e.g. radio or WLAN.

In the embodiment illustrated in FIG. 3, a screen 61 is provided as visualization unit 61, said screen having a camera 62, inter alia, as input means 62. As is directly evident from FIG. 3, a virtual reality is provided for the user 2 by means of the screen 61, said virtual reality showing the user 2 when performing a movement sequence, here lifting a weight. In this case, in the virtual surroundings, the weight is added as part of the virtual surroundings to the image of the user 2 recorded by the camera 61. In this case, the user's movement sequence with visualized weight is shown to the user 2 in real time. In accordance with FIG. 3, the system 1 here comprises a textile 10 in the form of a wing on which electrodes 8 and sensors 3 are arranged in each case on the back region of the upper arms. If the movement sequence stored in the data processing unit 4 is not performed correctly by the user 2, the user 2 receives a stimulation pulse via the electrodes 8. It is likewise possible to output a stimulation pulse as simulation of the game situation e.g. the effect of the lifted weight.

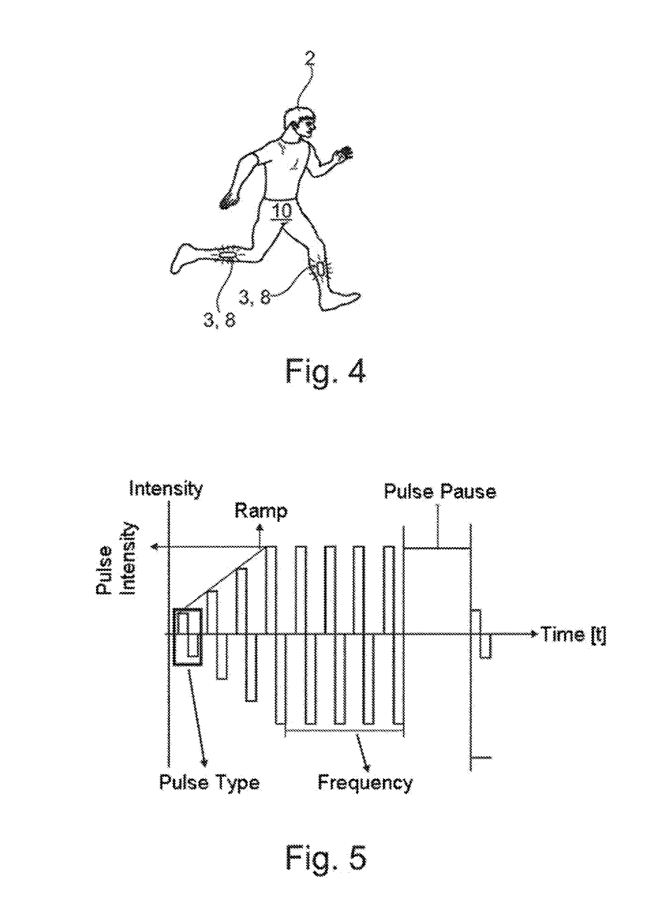

FIG. 4 shows an illustration for the control of stimulation pulses with an EMS user 2 who is equipped with at least two electrodes, here on training trousers 10. Said user is stimulated by pulses during his or her activity. The pulses are clocked by means of a sensor. A time, pressure, acceleration or ultrasonic sensor, resistance device or an electromyography device is optionally used in this case.

FIG. 5 shows an illustration of a voltage profile of an exemplary stimulation pulse. Such a stimulation pulse can be varied in one or more stimulation pulse parameters and triggered depending on the control signal, in particular by the pulse unit 5. In this case, it is directly evident from FIG. 5 that rectangular profiles of the pulse intensities are respectively present here. The entire stimulation pulse comprises a pulse unit composed of a plurality of individual pulses which are triggered with identical or different intensities in quick succession. In this case, each individual pulse is a single process whose instantaneous values deviate appreciably from zero only within a limited time period. The intensity of the stimulation pulse is attained after a sequence of ramp pulses increasing in their maximum excursion. The ramp, as illustrated in FIG. 5, exhibits here a gradient attained from the maximum excursions of the sequence of such ramp pulses increasing in their excursion. After the progression of the stimulation pulse, FIG. 5 illustrates a pulse pause, which denotes the temporal duration between two successive stimulation pulses. The stimulation pulse that succeeds the pulse pause is indicated on the basis of its first ramp pulse. The stimulation pulse illustrated has a pulse width of approximately 25 to approximately 200 μs.

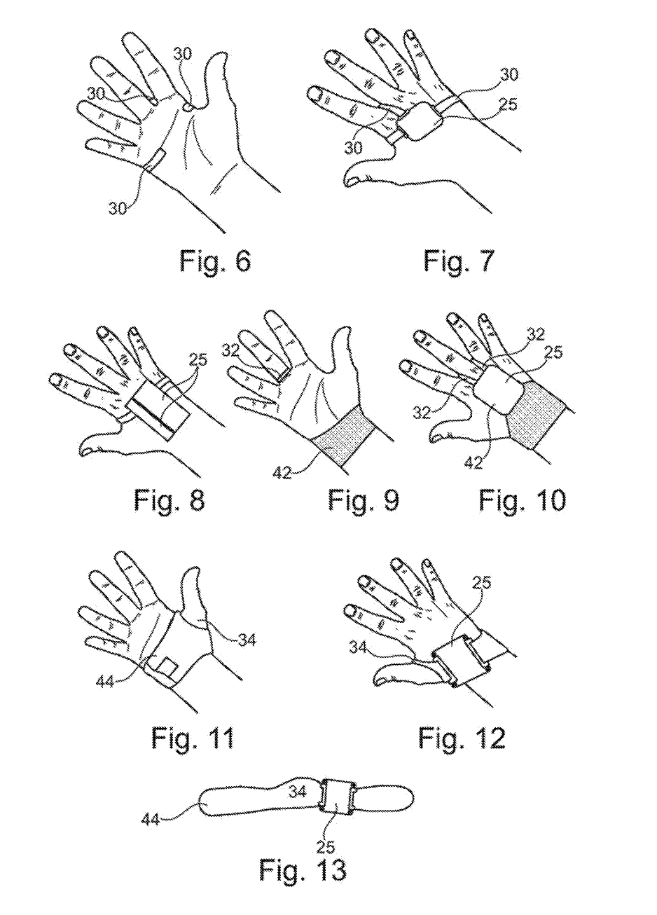

FIGS. 6 and 7 show an embodiment of a user interface 20 in the state where it is secured on a person's hand. The user interface 20 can perform the same tasks as, or different tasks than the user interface 6 already mentioned. It can be used as an alternative to the user interface 6 or in addition.

In this case, FIG. 6 shows the inside of the hand and FIG. 7 shows the outside of the hand. In accordance with FIG. 7, a communication module 25 is secured on the back of the hand. Said communication module can comprise input and output means, as will be described in detail later. Three holding clips 30 are secured on the communication module 25. Said holding clips are produced from a flexurally stiff material. This can be a thermoplastic or a metal clip sheathed, if appropriate, with plastic. Flexurally stiff means that it has a high mechanical restoring force vis-à-vis deformations. Furthermore, it can be plastically deformable. In other words, with a deformation beyond the yield point a user can give the holding clip 30 a new shape, in order to optimally adapt it to the geometry of the user's own hand. In the case of a holding clip 30 produced from plastic, the deformation can be carried out under the influence of temperature. A first holding clip engages into the thumb/index finger interspace. A further holding clip engages on the edge of the hand, that is to say that side of the main area of the hand which is opposite the thumb. The edge of the hand is the outer extension of the little finger in the direction of the flat of the hand. Moreover a third holding clip 30 can be in an arbitrary finger interspace. The holding clips are substantially U-shaped, wherein an 180° arc need not be complete. Even an at least 120° bend produces a certain holding function, the degree of swing preferably being at least 150°. Said arc is considered from the midpoint of the rounding.

FIG. 8 shows an alternative embodiment of the communication module 25. The latter is bipartite, wherein the two parts are connected to one another via an elastic connection, such as e.g. a plastic or textile band. Both parts comprise electronic components. Said components can be input regions, a display, a module in which a battery is accommodated, and/or a printed circuit board (PCB). During the movement of the hand, the back of the hand changes its shape. In order to enable the communication module 25 to bear well without play on the back of the hand, this flexibility is advantageous. The securing of the communication module 25 is realized by means of two holding clips in the example shown. Moreover, a continuous band can be led on the palm of the hand. It is also possible for the communication module 25 to comprise more than two components which are flexibly or elastically displaceable relative to one another. Moreover, electronic components are usable which are themselves bendable and/or elastically deformable.

FIGS. 9 and 10 show alternative embodiments of the user interface 20 comprising a wristband 42 and a finger loop 32. The wristband is embodied as a closed ring made from an elastic material, and the wristband 42 is held on the wrist on account of the restoring forces. Said band 42 is secured on the communication module 25. Furthermore, a finger loop 32 is attached there. The finger loop is an (elastic) band and embodied in the present case such that the middle finger can be passed through the band. In this regard, the communication module is held on the back of the hand between the finger loop 32 and the wristband 42 securely and without slipping.

A further embodiment is shown in FIGS. 11 to 13, where a band 44 for the ball of the thumb is used instead of a wristband 42. The band 44 for the ball of the thumb lies around the main surface of the hand in a ring-shaped fashion at the level of the ball of the thumb. Furthermore, a thumb loop 34 is provided, through which the thumb is inserted. In this regard, the communication module firstly is led in the longitudinal direction of the hand in a manner protected against displacement, and the band 44 for the ball of the thumb cannot twist either. The band 44 for the ball of the thumb is itself not a closed loop, but rather an elongate strip, as is evident from FIG. 13. There is a hook-and-loop connection at the ends, such that the user can easily put it on and adapt it to his/her individual size. A hook-and-loop connection can also be used in the case of the wristband in FIGS. 9 & 10, and equally a closed ring-shaped band (as in FIGS. 9 & 10) can be used in the case of a band for the ball of the thumb.

Preferably, the entire palm of the hand is not covered by the securing means. This allows the user to continue to use the sense of touch of his/her hand and e.g. to be able to grasp training devices well. In this sense, it is advantageous not to use a glove, nor a glove comprising shortened fingers or cut-off fingers, since there the palm of the hand is completely covered. In the case of the band for the ball of the thumb in FIGS. 11-13, although part of the palm of the hand is covered the desired touch capability is not restricted to an impermissibly great extent since the fingers and the wrist bones of the palm of the hand are not covered. Preferably, at least 50% of the palm of the hand is not covered by the securing means.

The following tasks can be performed by the communication module:

It can provide feedback about the training state. In this regard, in a detailed display it is possible to display information such as the training sequence or scope, such e.g. calories burnt. It is possible to output an overview of which muscle groups are being worked.

Provision can be made of feedback means that provide information about the next EMS pulse. By way of example, an EMS pulse can last for 3 seconds and then be followed by a pause of e.g. 3 seconds. In order that the next pulse is not a surprise for the user, an optical signal can be output via the user interface. In this regard, e.g. an LED can emit light or flash one second or half a second before the beginning of an EMS pulse. A haptic feedback is also possible. In this regard, a vibration can be exerted by the communication module 25. The hand is very sensitive and such vibrations are thus readily perceptible. Alongside the abovementioned output means, that is to say means which supply the user with information about the system state, input means can be provided. Via individual keys or a keypad, parameters of the stimulation such as pulse intensity, frequency, signal type (rectangular or sinusoidal) can be selected. Moreover, individual electrodes (or groups of electrodes) of the EMS can be chosen and activated.

Furthermore, it is possible to integrate a clock and/or a stopwatch in the communication module.

Moreover, an illumination can be provided, which is permanently switched on or can be switched on and off. In this case, said illumination is in particular not a function display that displays some state of the system of the EMS. Rather, said illumination, particularly in the case of outdoor use in the dark, can serve as a safety feature and make the training person visible e.g. in road traffic. The communication module can be embodied such that it is waterproof, in order not to be damaged in the rain in an outdoor application.

The communication module can also be inductively chargeable. A sporting activity often comprises an intensive arm movement. In the sense of energy harvesting, energy can be generated inductively. Stretching, compressing, movement, etc. are regarded as alternative principles.

### LIST OF REFERENCE SIGNS

1 system
2 user
3 sensor
4 data processing unit
5 pulse unit
6 user interface
61 visualization unit
62 input means
7 energy source
8 electrodes
9 conductor
10 textile
20 user interface
25 communication module
30 holding clip
32 finger loop
34 thumb loop
42 wristband
44 band for the ball of the thumb

What is claimed is:

1. A user interface, wherein
the user interface comprises: a communication module and securing means for securing the communication module on a back of a hand of a person training with the system, wherein at least 60%, in particular at least 80%, of a palm of the hand is not covered by the securing means, and
the communication module comprises at least one display for displaying an actual or a setpoint condition of an electrostimulation and/or input means for starting or ending an electrostimulation and/or for setting parameters of the electrostimulation and/or a display of at least one training-relevant parameter.

2. The user interface as claimed in claim 1, wherein the securing means comprise a thumb loop, through which a user can insert his/her thumb for securing the user interface.

3. The user interface as claimed in claim 1, wherein the securing means comprise at least one finger loop, through which a user can insert at least one finger for securing the user interface.

4. The user interface as claimed in claim 1, wherein the securing means comprise at least one holding clip, which is produced from a flexurally stiff material and is secured at least indirectly on the communication module and is configured in this case to encompass part of a flat of the hand in order to secure the user interface.

5. The user interface as claimed in claim 1, wherein the securing means comprise a wristband, which is guidable around the wrist in particular with an elastic press-on force and is preferably configured not to cover a ball of a thumb or to cover it only to an extent of 10%.

6. The user interface as claimed in claim 1, wherein the securing means comprise a band for the ball of the thumb, which is guidable around the hand in particular with an elastic press-on force.

7. The user interface as claimed in claim 5, wherein the wristband and/or the band for the ball of the thumb are/is designed such that they/it can open and close, and in this case comprise/comprises in particular a hook-and-loop connection.

8. The user interface as claimed in claim 5, wherein the wristband and/or the band for the ball of the thumb are/is produced from an elastic material and cannot be opened as a closed loop.

9. (canceled)

10. The user interface as claimed in claim 1, wherein the securing means are part of a garment for covering the forearm of a person using the user interface and/or the communication module is connectable via the securing means, in particular a hook-and-loop connection, to a garment for covering the forearm of a person using the user interface, wherein said garment is preferably a shirt having a thumb loop and/or finger loop.

11. The user interface as claimed in claim 1, wherein the communication module comprises at least two electronic components, such as, in particular a display, input switch, output luminaire(s), such as, in particular, LED, and/or an energy supply, and a relative movement, in particular a pivoting movement, of these components with respect to one another is possible.

12. The user interface as claimed in claim 1, wherein the user interface is not a glove, not a mitten and not a fingerless glove.

13. The user interlace as claimed in claim 2, wherein a band runs over the flat of the hand and the back of the hand and the thumb loop fixes the band.

14. The user interface as claimed in claim 1, wherein the user interface comprises a control unit and/or is connectable to the control unit and the control unit is configured to activate signals of electro-muscle stimulation and/or vary parameters of the electro-muscle stimulation.

15. The user interface as claimed in claim 1, wherein the control unit comprises an inclination sensor, a compass and/or an acceleration sensor and is configured to measure angular positions and/or movements and/or accelerations and to identify inputs or commands of the user from these measurement values in the sense of gesture control.

16. The user interface as claimed in claim 1, wherein the communication module can comprise haptic feedback means for generating mechanical, thermal or electrical stimuli.
